(19) United States
(12) Patent Application Publication
(10) Pub. No.: US 2013/0190236 A1
(43) Pub. Date: Jul. 25, 2013

(54) BMP-7 Variants with Reduced Immunogenicity

(71) Applicant: STRYKER CORPORATION, Kalamazoo, MI (US)

(72) Inventors: Marilyn Elizabeth Patterson, Leicester, MA (US); Moulay Hicham Alaoui-Ismaili, San Mateo, CA (US)

(73) Assignee: STRYKER CORPORATION, Kalamazoo, MI (US)

(21) Appl. No.: 13/766,014

(22) Filed: Feb. 13, 2013

Related U.S. Application Data
(63) Continuation of application No. 13/517,366, filed on Feb. 12, 2013, now abandoned.
(60) Provisional application No. 61/289,220, filed on Dec. 22, 2009.

(51) Int. Cl. C07K 14/51

(52) U.S. Cl. CPC C07K 14/51
USPC 514/8.8; 530/399; 536/23.5; 435/320.1; 435/243; 435/69.1

(57) ABSTRACT

The invention is directed to bone morphogenetic proteins that have reduced immunogenicity. In particular, the invention is directed to human BMP-7 that has been modified to reduce immunogenicity through alteration of the amino acid sequence of wild-type BMP-7.

### CROSS-REFERENCE TO PRIOR APPLICATIONS

This application claims priority to and the benefit of U.S. Provisional Patent Application No. 61/289,220, filed Dec. 22, 2009, the contents of which is incorporated by reference herein.

### TECHNICAL FIELD OF THE INVENTION

This invention is related to Bone Morphogenetic Protein-7 (BMP-7) that has been modified to reduce immunogenicity and methods for modifying BMP-7 to reduce immunogenicity.

### BACKGROUND

BMP-7, also known as Osteogenic Protein-1 (OP-1), a protein capable of inducing bone growth, is useful for treating a variety of cartilage and bone disorders and defects. For example, recombinant human BMP-7 has been used to treat over 40,000 patients globally. However, clinical results have revealed that recombinant human BMP-7 is highly immunogenic in some clinical indications. In other words, the recombinant protein can stimulate an immune response in a patient, causing the patient to develop antibodies against BMP-7. These antibodies can also inhibit function of BMP-7 produced endogenously by the patient, resulting in potential long-term consequences for patient health. Accordingly, there is a need in the art for BMP-7, including recombinant BMP-7, having reduced immunogenicity in order to improve its effectiveness and reduce adverse effects in patients, while maintaining its biological activity and clinically relevant bone morphogenetic properties.

### SUMMARY OF THE INVENTION

The present invention is directed to BMP-7, for example human recombinant BMP-7, which has been modified to reduce its immunogenicity in comparison to wild-type human BMP-7. More specifically, the BMP-7 proteins according to the invention are modified to remove potential immunogenic epitopes. As a result, BMP-7 proteins of the invention have improved biological properties as compared to wild-type BMP-7.

According to one aspect, the invention includes a variant BMP-7 protein having at least 90% sequence identity with mature human BMP-7. The variant BMP-7 contains substitutions at one or more, two or more, three or more, four or more, five or more, six or more, seven or more, or eight or more of the following positions corresponding to mature human BMP-7: G61, A63, Y65, Y66, E68, E70, A72, H92, F93, I94, N95, P96, E97, T98, V99, P100, P102, or A105. In a further embodiment, the variant protein has at least 95% identity with mature human BMP-7.

In a further embodiment the substitutions are one or more of the following: G61E, A63E/Q/H, Y65F/N, Y66E, E68D/K/H, E70G/D, A72S/F/P, H92N, F93N/L/S, 194M/K/SN, N95V/F, P96S/N, E97S/T/D/K, T98K/I/S/Y/H, V99I, P100G, P102A, or A105V. In a further embodiment, the variant demonstrates BMP-7 activity.

In another aspect, the invention is directed to a nucleic acid encoding a variant BMP-7 protein of the invention. For example, the nucleic acid is DNA or RNA.

In another aspect, the invention is directed to a recombinant expression vector containing a nucleic acid encoding the variant BMP-7 protein of the invention.

In yet another aspect, the invention is directed to a cell containing an expression vector containing a nucleic acid encoding the variant BMP-7 protein of the invention. The cell may be prokaryotic in one embodiment, or eukaryotic in another embodiment.

In a further aspect, the invention is directed to a pharmaceutical composition that includes a variant BMP-7 protein of the invention and a pharmaceutical carrier.

According to a further aspect, the invention is directed to a method of treating a skeletal disorder in a patient. The method requires administering to the patient a therapeutically effective amount of a variant BMP-7 protein of the invention.

According to yet a further aspect, the invention is directed to a method of reducing the immunogenicity of a human BMP-7 protein. The method requires identifying an immunogenic epitope on human BMP-7 and modifying the epitope in the amino acid sequence of human BMP-7 by engineering one or more substitutions in the amino acid sequence of BMP-7 to create a modified amino acid sequence. The one or more substitutions occurs at one or more of positions G61, A63, Y65, Y66, E68, E70, A72, H92, F93, I94, N95, P96, E97, T98, V99, P100, P102, or A105 corresponding to mature human BMP-7. In one embodiment, the human BMP-7 is recombinant. In a further embodiment, the one or more substitutions is any one or more of G61E, A63E/Q/H, Y65F/N, Y66E, E68D/K/H, E70G/D, A72S/F/P, H92N, F93N/L/S, 194M/K/SN, N95V/F, P96S/N, E97S/T/D/K, T98K/I/S/Y/H, V99I, P100G, P102A, or A105V. In a further embodiment, the method can include the steps of expressing a protein encoded by the modified amino acid sequence in a suitable expression system and purifying the protein. The protein may be expressed in a eukaryotic cell in one embodiment, or a prokaryotic cell in another embodiment.

### BRIEF DESCRIPTION OF THE FIGURES

FIG. 1 shows eighteen peptides covering the entire sequence of the mature region of human BMP-7, each fifteen amino acids long, and overlapping by either 5 or 10 amino acids.

FIG. 2 is a schematic representation of the eighteen peptides showing the overlap between these peptides in relation to the entire mature region of human BMP-7.

FIG. 3 is a bar graph showing the results of binding in an ELISA of the eighteen peptides shown in FIG. 1 to non-neutralizing anti-BMP-7 antibodies from patient serum samples. (The bars are from left to right, 111266−, 111694+, 111945+, 111665+, and 1B12/12G3+).

FIG. 4 is a bar graph showing the results of binding in an ELISA of the eighteen peptides shown in FIG. 1 to neutralizing anti-BMP-7 antibodies from patient serum samples. (the bars are from left to right 113791−, 113113+, 113331+, 113756+, 112956+, 113757+, 113766+, 111694+, and 1B12/12G3+).

FIG. 5A is an alignment of the portions of BMP-7 corresponding to peptide 9 (as shown in FIG. 1) with the corresponding regions in BMP-2, 4, 5, 6, and 9.

FIG. 5B is an alignment of the portions of BMP-7 corresponding to peptide 13 (as shown in FIG. 1) with the corresponding regions in BMP-2, 4, 5, 6, and 9.

FIG. 6 is the sequence of mature human BMP-7 (SEQ ID NO:1).

### DETAILED DESCRIPTION OF THE INVENTION

Recombinant human BMP-7 has been shown to be highly immunogenic in some clinical indications. For example, BMP-7, when implanted in patients as part of OP-1® Putty or OP-1® Implant (Stryker Biotech Hopkinton, Mass.), causes some patients to exhibit an immune response by generating antibodies to recombinant human BMP-7. This reduces the effectiveness of the BMP-7 treatment and can lead to side effects.

Accordingly, the invention is directed to variant BMP-7 proteins that have reduced immunogenicity as compared to wild-type BMP-7. The invention also includes methods of making and using BMP-7 variants with reduced immunogenicity. Immunogenicity is reduced, according to the invention, by modifying the amino acid residues of BMP-7 moieties containing potential immunogenic epitopes. Accordingly, BMP-7 proteins modified according to the invention maintain their biological activity, but are substantially less immunogenic than their wild type BMP-7 counterpart. For example, the immunogenic properties of BMP-7 are eliminated or substantially reduced according to the invention. Accordingly, it is expected that such variant BMP-7 proteins will be less immunogenic when administered to patients, e.g., human patients.

### Bone Morphogenetic Proteins

Bone morphogenetic proteins (BMPs) belong to the TGF-β superfamily. The TGF-β superfamily proteins are cytokines characterized by six-conserved cysteine residues. The human genome contains about 42 open reading frames encoding TGF-β superfamily proteins. The TGF-β superfamily proteins can at least be divided into the BMP subfamily and the TGF-β subfamily based on sequence similarity and the specific signaling pathways that they activate. The BMP subfamily includes, but is not limited to, BMP-2, BMP-3 (osteogenin), BMP-3b (GDF-10), BMP-4 (BMP-2b), BMP-5, BMP-6, BMP-7 (osteogenic protein-1 or OP-1), BMP-8 (OP-2), BMP-8B (OP-3), BMP-9 (GDF-2), BMP-10, BMP-11 (GDF-11), BMP-12 (GDF-7), BMP-13 (GDF-6, CDMP-2), BMP-15 (GDF-9), BMP-16, GDF-1, GDF-3, GDF-5 (CDMP-1, MP-52), and GDF-8 (myostatin). Furthermore, there is allelic variation in BMP sequences among different members of the human population, and there is species variation among BMPs discovered and characterized to date. As used herein, “BMP subfamily,” “BMPs,” “BMP ligands,” and grammatical equivalents thereof refer to the BMP subfamily members, unless specifically indicated otherwise.

Publications disclosing these sequences, as well as their chemical and physical properties, include: BMP-7 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)), BMP-2, BMP-4, (WO88/00205; Wozney et al. Science, 242, pp. 1528-1534 (1988)), BMP-5 and BMP-6, (Celeste et al., PNAS, 87, 9843-9847 (1990)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/US95/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/US94/00657) and WO96/14335 (PCT/US94/12814) and WO93/16099 (PCT/EP93/00350)); GDF-6 (CDMP-2, BMP13) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)) The above publications are incorporated herein by reference.

As used herein, “TGF-β superfamily member” or “TGF-β superfamily protein,” means a protein known to those of ordinary skill in the art as a member of the Transforming Growth Factor-β (TGF-β) superfamily. Structurally, such proteins are homo or heterodimers expressed as large precursor polypeptide chains containing a hydrophobic signal sequence, an N-terminal pro region of several hundred amino acids, and a mature domain comprising a variable N-terminal region and a highly conserved C-terminal region containing approximately 100 amino acids with a characteristic cysteine motif having a conserved six or seven cysteine skeleton. These structurally-related proteins have been identified as being involved in a variety of developmental events.

The term “morphogenic protein” refers to a protein belonging to the TGF-β superfamily of proteins which has true morphogenic activity. For instance, such a protein is capable of inducing progenitor cells to proliferate and/or to initiate a cascade of events in a differentiation pathway that leads to the formation of cartilage, bone, tendon, ligament, neural or other types of differentiated tissue, depending on local environmental cues. Thus, morphogenic proteins useful according to the invention can behave differently in different surroundings. In certain embodiments, a morphogenic protein of this invention can be a homodimer species or a heterodimer species.

The term “osteogenic protein (OP)” refers to a morphogenic protein that is also capable of inducing a progenitor cell to form cartilage and/or bone. The bone can be intramembranous bone or endochondral bone. Most osteogenic proteins are members of the BMP subfamily and are thus also BMPs. However, the converse can not be true. According to this invention, a BMP identified by DNA sequence homology or amino acid sequence identity must also have demonstrable osteogenic or chondrogenic activity in a functional bioassay to be an osteogenic protein. Appropriate bioassays are well known in the art; a particularly useful bioassay is the heterotopic bone formation assay (see, U.S. Pat. No. 5,011,691; U.S. Pat. No. 5,266,683, for example).

Structurally, BMPs are dimeric cysteine knot proteins. Each BMP monomer comprises multiple intramolecular disulfide bonds. An additional intermolecular disulfide bond mediates dimerization in most BMPs. BMPs can form homodimers. Some BMPs can form heterodimers. BMPs are expressed as pro-proteins comprising a long pro-domain, one or more cleavage sites, and a mature domain. The pro-domain is believed to aid in the correct folding and processing of BMPs. Furthermore, in some but not all BMPs, the pro-domain can non-covalently bind the mature domain and can act as an inhibitor (e.g., Thies et al., (2001) Growth Factors 18:251-259).

BMPs are naturally expressed as pro-proteins comprising a long pro-domain, one or more cleavage sites, and a mature domain. This pro-protein is then processed by the cellular machinery to yield a dimeric mature BMP molecule. The pro-domain is believed to aid in the correct folding and processing of BMPs. Furthermore, in some but not all BMPs, the pro-domain can noncovalently bind the mature domain and can act as a chaperone, as well as an inhibitor (e.g., Thies et. al., (2001) Growth Factors, 18:251-259).

BMP signal transduction is initiated when a BMP dimer binds two type I and two type II serine/threonine kinase receptors. Type I receptors include, but are not limited to, ALK-1, ALK-2 (also called ActR1a or ActRI), ALK-3 (also called BMPRIa), and ALK-6 (also called BMPRIb). Type II receptors include, but are not limited to, ActRIIa (also called ActRII), ActRIIb, and BMPRII. Human genome contains 12 members of the receptor serine/threonine kinase family, including 7 type I and 5 type II receptors, all of which are involved in TGF-β signaling (Manning et al., 2002, Science, 298:1912-1934) the disclosures of which are hereby incorporated by reference). Following BMP binding, the type II receptors phosphorylate the type I receptors, the type I receptors phosphorylate members of the Smad family of transcription factors, and the Smads translocate to the nucleus and activate the expression of a number of genes.

BMPs also interact with inhibitors, soluble receptors, and decoy receptors, including, but not limited to, BAMBI (BMP and activin membrane bound inhibitor), BMPER (BMP-binding endothelial cell precursor-derived regulator), Cerberus, cordin, cordin-like, Dan, Dante, follistatin, follistatin-related protein (FSRP), ectodin, gremlin, noggin, protein related to Dan and cerberus (PRDC), sclerostin, sclerostin-like, and uterine sensitization-associated gene-1 (USAG-1). Furthermore, BMPs can interact with co-receptors, for example BMP-2 and BMP-4 bind the co-receptor DRAGON (Samad et. al. (2005) J. Biol. Chem., 280:14122-14129), and extracellular matrix components such as heparin sulfate and heparin (Irie et al. (2003) Biochem. Biophys. Res. Commun. 308: 858-865).

As contemplated herein, the term “BMP” refers to a protein belonging to the BMP subfamily of the TGF-β superfamily of proteins defined on the basis of DNA homology and amino acid sequence identity. According to this invention, a protein belongs to the BMP subfamily when it has at least 50% amino acid sequence identity with a known BMP subfamily member within the conserved C-terminal cysteine-rich domain that characterizes the BMP subfamily. Members of the BMP subfamily can have less than 50% DNA or amino acid sequence identity overall. As used herein, the term “BMP” further refers to proteins which are amino acid sequence variants, domain-swapped variants, and truncations and active fragments of naturally occurring bone morphogenetic proteins, as well as heterodimeric proteins formed from two different monomeric BMP peptides, such as BMP-2/7; BMP-4/7: BMP-2/6; BMP-2/5; BMP-4/7; BMP-4/5; and BMP-4/6 heterodimers. Suitable BMP variants and heterodimers include those set forth in US 2006/0235204; WO 07/087,053; WO 05/097825; WO 00/020607; WO 00/020591; WO 00/020449; WO 05/113585; WO 95/016034 and WO93/009229.

According to one embodiment, a BMP variant, such as a BMP-7 variant, created according to the methods of the invention maintains at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with the corresponding wild-type BMP protein sequence.

According to one embodiment, a BMP variant, such as a BMP-7 variant, created according to the methods of the invention maintains at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with the conserved cysteine domain of the C-terminal region of the corresponding wild-type BMP protein sequence.

By “corresponding wild-type protein” it is meant the wild-type version of the modified or variant BMP. For example, if the modified or variant BMP is a modified or variant BMP-7, the corresponding wild-type BMP is wild-type BMP-7.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=# of identical positions/total # of positions×100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research 25 (17):3389-3402. When utilizing BLAST and Gapped BLAST programs, the default parameters of the respective programs (e.g., XBLAST and NBLAST) can be used.

### Variant BMP-7s

The invention provides methods for reducing the immunogenicity of BMP-7. In order to reduce or eliminate the immunogenicity, variant BMP-7 proteins are created that differ from wild-type BMP-7. According to an embodiment of the invention, these variants differ from wild-type BMP-7 in that one or more amino acids in an immunogenic epitope of BMP-7 is modified. According to an embodiment of the invention, potential immunogenic epitopes of BMP-7 are identified as described herein and/or according to other methods known in the art and the epitopes are modified to reduce or eliminate the immunogenic effect of the epitope. Amino acid modifications, such as substitutions, deletions, or insertions, are then made in the epitopic regions according to standard genetic engineering procedures to reduce or eliminate the immunogenic effect of the epitope. According to one embodiment of the invention, BMP-7 variants of the invention maintain their bioactivity, while they have reduced or substantially reduced or eliminated immunogenicity in comparison to wild-type BMP-7.

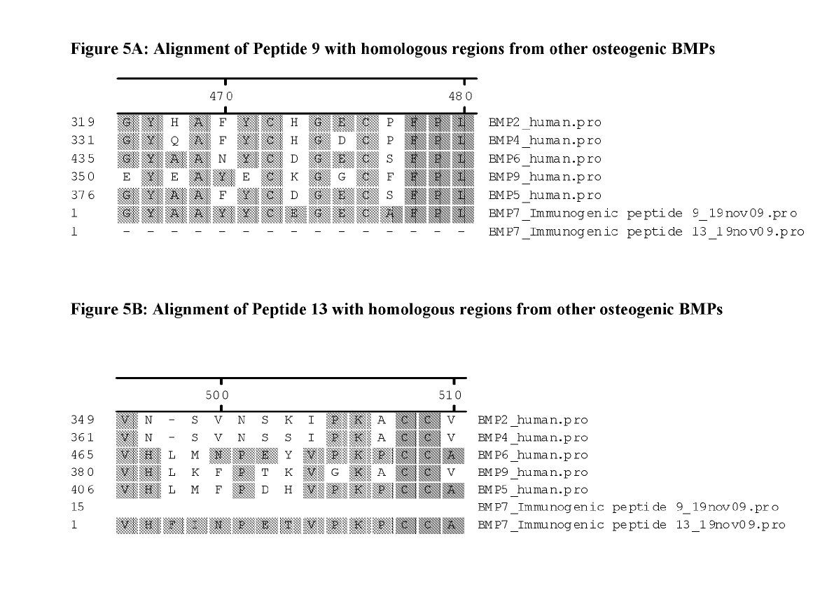

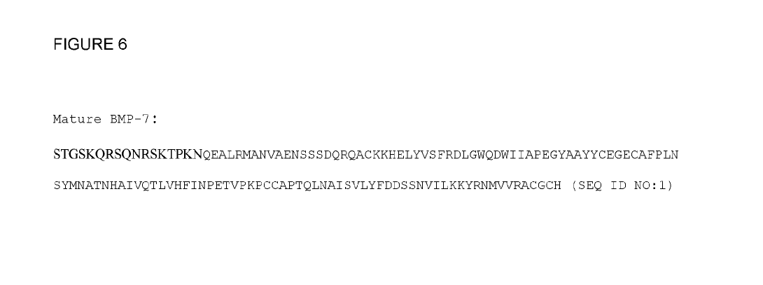

According to one embodiment of the invention, a region of BMP-7 identified as containing an epitope is replaced with the amino acid sequence from the corresponding region of another BMP in order to remove the epitope. For example, in one embodiment, the sequence of mature human BMP-7 from residue 61-75 is replaced with the corresponding amino acid sequence from BMP-2 (GYHAFYCHGECPFPL (SEQ ID NO:20)—residues 319-333 of FIG. 5A), BMP-4 (GYQAFYCHGDCPFPL (SEQ ID NO:21)—residues 331-345 of FIG. 5A), BMP-5 (GYAAFYCDGECSFPL (SEQ ID NO:22)—residues 376-390 of FIG. 5A), BMP-6 (GYAANYCDGECSFPL (SEQ ID NO:23)—residues 435-449 of FIG. 5A), or BMP-9 (EYEAYECKGGCFFPL (SEQ ID NO:24)—residues 350-364 of FIG. 5A). In another embodiment, the sequence of mature human BMP-7 from residue 91-105 is replaced with the corresponding amino acid sequence from BMP-2 (VNSVNSKIPKACCV (SEQ ID NO:25)—residues 349-362 of FIG. 5B), BMP-4 (VNSVNSSIPKACCV (SEQ ID NO:26)—residues 361-374 of FIG. 5B), BMP-5 (VHLMFPDHVPKPCCA (SEQ ID NO:27)—residues 406-420 of FIG. 5B), BMP-6 (VHLMNPEYVPKPCCA (SEQ ID NO:28)—residues 465-479 of FIG. 5B), or BMP-9 (VHLKFPTKVGKACCV (SEQ ID NO:29)—residues 380-394 of FIG. 5B).

In another embodiment of the invention, one or more amino acids of BMP-7 identified as being in a region of BMP-7 containing an epitope is modified, by substitution for example, of an amino acid corresponding to that residue in another BMP in order to remove the epitope. For example, as shown in FIGS. 5A and 5B, an alignment of two putative epitopic regions (Peptides 9 and 13) with corresponding regions from other BMP proteins is shown, suggesting possible amino acid modifications to BMP-7.

In another embodiment of the invention, one or more point mutations is introduced into human BMP-7 to remove an epitope. For example, in one embodiment, BMP-7 has a substitution at one or more of residues G61, A63, Y65, Y66, E68, E70, A72, H92, F93, I94, N95, P96, E97, T98, V99, P100, P102, or A105.

In another embodiment, BMP-7 has a substitution at two or more of residues G61, A63, Y65, Y66, E68, E70, A72, H92, F93, I94, N95, P96, E97, T98, V99, P100, P102, or A105.

In another embodiment, BMP-7 has a substitution at three or more of residues G61, A63, Y65, Y66, E68, E70, A72, H92, F93, I94, N95, P96, E97, T98, V99, P100, P102, or A105.

In another embodiment, BMP-7 has a substitution at four or more of residues G61, A63, Y65, Y66, E68, E70, A72, H92, F93, I94, N95, P96, E97, T98, V99, P100, P102, or A105.

In another embodiment, BMP-7 has a substitution at five or more of residues G61, A63, Y65, Y66, E68, E70, A72, H92, F93, I94, N95, P96, E97, T98, V99, P100, P102, or A105.

In another embodiment, BMP-7 has a substitution at six or more of residues G61, A63, Y65, Y66, E68, E70, A72, H92, F93, I94, N95, P96, E97, T98, V99, P100, P102, or A105.

In another embodiment, BMP-7 has a substitution at seven or more of residues G61, A63, Y65, Y66, E68, E70, A72, H92, F93, I94, N95, P96, E97, T98, V99, P100, P102, or A105.

In another embodiment, BMP-7 has a substitution at eight or more of residues G61, A63, Y65, Y66, E68, E70, A72, H92, F93, I94, N95, P96, E97, T98, V99, P100, P102, or A105.

In another embodiment, BMP-7 has a substitution at nine or more of residues G61, A63, Y65, Y66, E68, E70, A72, H92, F93, I94, N95, P96, E97, T98, V99, P100, P102, or A105.

In another embodiment, BMP-7 has a substitution at ten or more of residues G61, A63, Y65, Y66, E68, E70, A72, H92, F93, I94, N95, P96, E97, T98, V99, P100, P102, or A105.

In a further embodiment, BMP-7 has one or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has two or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has three or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has four or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has five or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has six or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has seven or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has eight or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has nine or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

In a further embodiment, BMP-7 has ten or more of the following substitutions: G61E, A63E/Q/H, Y65F/N, Y66E, E68D/K/H, E70D/G, A72S/F/P, H92N, F93N/L/S, 194M/K/SN, N95V/F, P96S/N, E97S/T/D/K, T98K/I/S/Y/H, V99I, P100G, P102A, or A105V.

According to another aspect of the invention, BMP-7 variants according to the invention maintain BMP-7 biological activity. As used herein, the term “biological activity” refers to any measurable function of BMPs in vivo or in vitro. Some of the ways in which biological activity of BMPs can be measured are listed in the “Examples” section below. In one embodiment, a BMP-7 variant of the invention has at least 30%, at least 40%, at least 50%, at least 55%, at least 60%, at least 65%, at least 70%, at least 75%, at least 80%, at least 85%, at least 90%, at least 95%, at least 98%, at least 99% of the biological activity as compared to wild-type BMP-7. For example, in one embodiment, a BMP-7 variant of the invention has at least one of the aforementioned % biological activity as compared to human wild-type BMP-7 or recombinant human wild-type BMP-7.

### Therapeutic Uses of BMP-7 Variants

BMP-7 variants according to the invention can be implanted in or administered to a mammalian patient, for example, a human to treat a wide variety of conditions. BMP-7 variants of the invention can alone or in combination with an appropriate carrier or other formulation agents be implanted in solid, gel or paste form, or injected into the patient in a gel, paste or liquid form. BMP-7 variants of the invention are useful for treating a wide variety of conditions. For example, BMP-7 variants of the invention can be used to treat skeletal disorders, including cartilage degeneration whether caused by trauma or inflammatory disease. For example, diseases treatable by BMP-7 variants of the invention include rheumatoid arthritis (RA) and osteoarthritis (OA) and autoimmune diseases such as systemic lupus erythematosis (SLE) and scleroderma.

The BMP-7 variants of the invention can be used effectively to treat skeletal diseases or injuries. For example, the BMP-7 variants can be used to treat a bone fracture, such as an open fracture or a closed fracture. For the treatment of a closed fracture, the BMP-7 variant is preferably injected at the fracture site. For open fractures, critical size defects or persistent nonunions, the BMP-7 variant can be administered by surgical implantation at the fracture site. In both cases, the BMP-7 variant can be administered alone, or in combination with a suitable carrier, matrix or scaffold, such as a bone cement, a calcium phosphate material, a gel material or a collagen matrix. Suitable carriers, matrices and scaffolds include those disclosed in U.S. Pat. Nos. 6,919,308; 6,949,251; and 7,041,641.

In a preferred embodiment, the BMP-7 variants of the invention can be used to treat a disease or injury resulting in cartilage degradation or a cartilage defect. For example, BMP-7 variants of the invention can be applied to a cartilage defect site, such as a degenerative intervertebral disc, or other fibrocartilaginous tissue, including a tendon, a ligament or a meniscus. Such methods are set out in U.S. Pat. No. 6,958,149. The BMP-7 variants of the invention can also be used to treat a defect or degeneration of articular cartilage, as set forth in published PCT application WO 05/115438, such as the cartilage lining of a joint, such as a synovial joint, including a knee, an elbow, a hip, or a shoulder. In this embodiment, the BMP-7 variant is preferably injected into the synovial space of the joint. In another embodiment, the BMP-7 variant of the invention is used to treat an articular cartilage defect site, such as a chondral defect or an osteochondral defect, in a joint. Such articular cartilage defects can be the result of a disease process, such as osteoarthritis or rheumatoid arthritis, or due to injury of the joint. In this embodiment, the BMP-7 variant can be injected into the joint space or it can be surgically implanted. For example, the BMP-7 variant can be placed within the defect either alone or in combination with one or more additional active agents, a supporting matrix or scaffold, or marrow stromal cells. The BMP-7 variant, one placed within the defect can, optionally, be covered with a suitable covering, for example a muscle flap or a bioresorbable membrane, such as a collagen membrane.

As will be appreciated by those skilled in the art, the concentration of BMP-7 variant to be administered to a patient will vary depending upon a number of factors, including without limitation the dosage of the drug to be administered and the route of administration. The preferred dosage of drug to be administered also is likely to depend on variables including, but not limited to, the type and extent of a disease, tissue loss or defect, the overall health status of the particular patient, the relative biological efficacy of the compound selected, the formulation of the compound, the presence and types of excipients in the formulation, and the route of administration. The BMP-7 variants of the present invention can be provided to an individual where typical doses range from about 10 ng/kg to about 1 g/kg of body weight per day; with a preferred dose range being from about 0.1 mg/kg to 100 mg/kg of body weight, and with a more particularly preferred dosage range of 10-1000 μg/dose. In a particularly preferred embodiment, a dose of 10-1000 μg of a BMP-7 is administered to an individual afflicted with osteoarthritis.

Additionally, as described below, BMP-7 variants of the present invention can be used to treat diseases or injuries of non-skeletal tissues. As further contemplated by the present invention, BMPs are capable of inducing the developmental cascade of bone morphogenesis and tissue morphogenesis for a variety of tissues in mammals different from bone or bone cartilage. This morphogenic activity includes the ability to induce proliferation and differentiation of progenitor cells, and the ability to support and maintain the differentiated phenotype through the progression of events that results in the formation of bone, cartilage, non-mineralized skeletal or connective tissues, and other adult tissues.

For example, BMP-7 variants of the invention can be used for treatment to prevent loss of and/or increase bone mass in metabolic bone diseases. General methods for treatment to prevent loss of and/or increase bone mass in metabolic bone diseases using osteogenic proteins are disclosed in U.S. Pat. No. 5,674,844, the disclosures of which are hereby incorporated by reference. BMP-7 variants of the present invention can be used for periodontal tissue regeneration. General methods for periodontal tissue regeneration using osteogenic proteins are disclosed in U.S. Pat. No. 5,733,878, the disclosures of which are hereby incorporated by reference. BMP-7 variants can be used for liver regeneration. General methods for liver regeneration using osteogenic proteins are disclosed in U.S. Pat. No. 5,849,686, the disclosures of which are hereby incorporated by reference. BMP-7 variants can be used for treatment of chronic renal failure. General methods for treatment of chronic renal failure using osteogenic proteins are disclosed in U.S. Pat. No. 6,861,404, the disclosures of which are hereby incorporated by reference. BMP-7s of the invention can be used for enhancing functional recovery following central nervous system ischemia or trauma. General methods for enhancing functional recovery following central nervous system ischemia or trauma using osteogenic proteins are disclosed in U.S. Pat. No. 6,407,060, the disclosures of which are hereby incorporated by reference. BMP-7 variants of the invention can be used for inducing dendritic growth. General methods for inducing dendritic growth using osteogenic proteins are disclosed in U.S. Pat. No. 6,949,505, the disclosures of which are hereby incorporated by reference. BMP-7 variants can be used for inducing neural cell adhesion. General methods for inducing neural cell adhesion using osteogenic proteins are disclosed in U.S. Pat. No. 6,800,603, the disclosures of which are hereby incorporated by reference. BMP-7 variants can be used for treatment and prevention of Parkinson's disease. General methods for treatment and prevention of Parkinson's disease using osteogenic proteins are disclosed in U.S. Pat. No. 6,506,729, the disclosures of which are hereby incorporated by reference.

As another example, BMP-7 variants can also be used to induce dentinogenesis. To date, the unpredictable response of dental pulp tissue to injury is a basic clinical problem in dentistry. As yet another example, BMP-7 variants can induce regenerative effects on central nervous system (CNS) repair can be assessed using a rat brain stab model.

### Example 1

### Identifying Immunogenic Epitopes Via ELISA

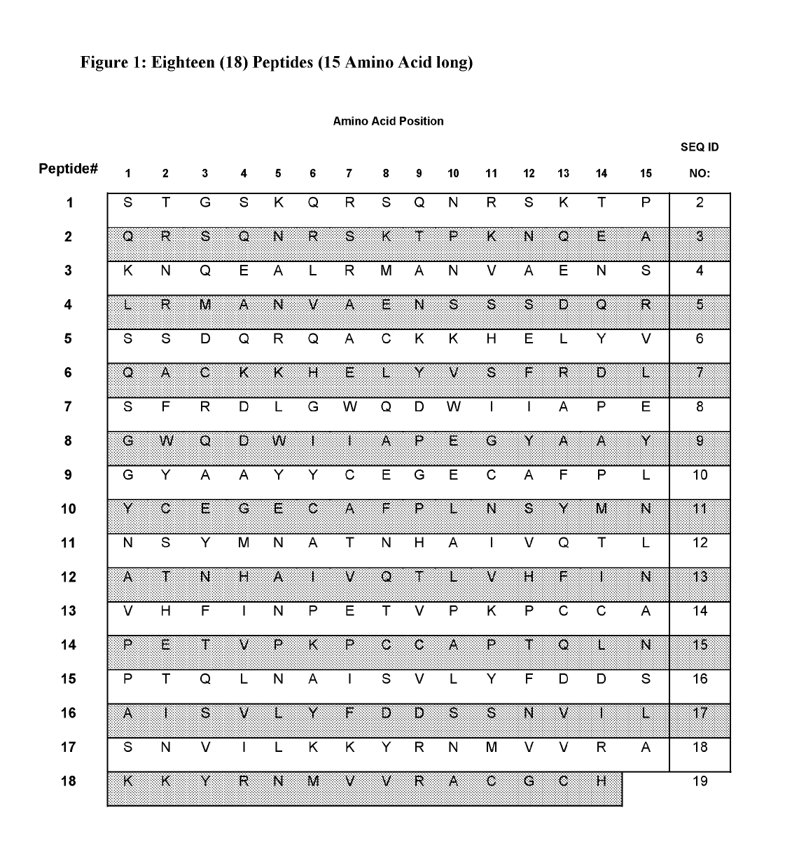

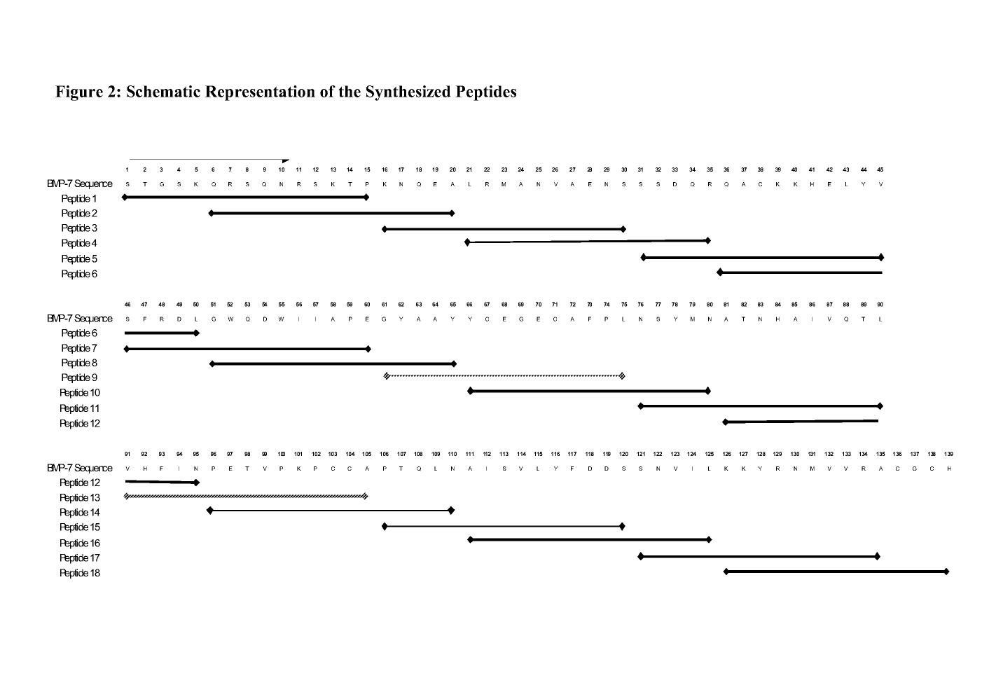

In order to identify potential linear T-cell epitopes of BMP-7, peptides covering the entire sequence of the mature region of wild-type human BMP-7 were synthesized (Synthetic Biomolecules San Diego, Calif.). Eighteen (18) peptides of fifteen (15) amino acids each were constructed. Each peptide had an overlap of 5 to 10 amino acids with peptides covering contiguous regions of BMP-7. The sequence of each of the 18 peptides is shown in FIG. 1 and the overlap between the various peptides is shown in FIG. 2.

The 18 peptides were tested in an ELISA assay to determine their binding to anti-BMP-7 antibodies. Each of the 18 peptides was individually coated on an individual row of a 96 well high-binding microtiter plate at a concentration of 5 μg/mL. Three plates were used with plate one having peptides 1-6, plate 2 having peptides 7-12, and plate 3 having peptides 13-18. A synthetically produced negative control peptide was coated on each plate in row 7 to serve as a negative control. BMP-7 was coated on each plate in row 8 to serve as a positive control.

The coated plates were incubated at room temperature overnight. The next day the plates were washed six times in BBS/T. The plates were then blocked with 200 μl/well of BBS/T milk and incubated at 37° C. for 2 hours. The plates were again washed six times with BBS/T.

Seven patient serum samples positive for neutralizing antibodies to BMP-7 and three patient serum samples positive for non-neutralizing antibodies to BMP-7 were diluted 1:80 in BBS/T milk and added to two adjacent columns of all 3 plates (tested in duplicates at 100 μl/well). The patient serum samples were obtained from patients treated with OP-1 Putty (Stryker Biotech Hopkinton, Mass.). For example, serum from patient 1 was added to columns 1-2 of all plates, serum from patient 2 was added to columns 3-4 of all plates and so on. One patient serum sample negative for anti-BMP-7 antibodies was used as a negative control. A combination of monoclonal anti-BMP-7 antibodies, 1B12 and 12G3, was used as a positive control.

Patient serum samples were added to peptide-coated plates and incubated for 1 hour at 37° C. The plates were washed six times with BBS/T. 100 μl of Goat anti-Human Ig HRP (Southern Biotech) was added to each well at a dilution of 1:40,000 in BBS/T milk. The plates were subsequently incubated for 1 hour at 37° C. and then washed six times in BBS/T milk. 100 μl of TMB substrate (BioFX) was added to each well for development.

100 μl of 0.18 M H₂SO₄ sulfuric acid stop solution was added to each plate. The plates were then placed in the M5 SpectraMax (Molecular Devices) and read at 450 nm for Optical Density.

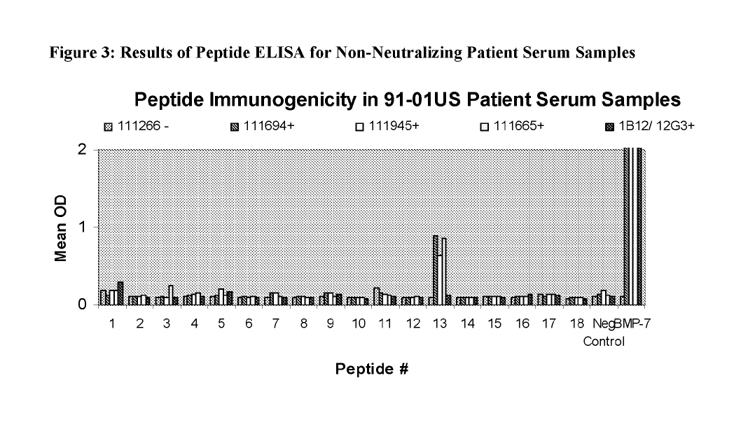

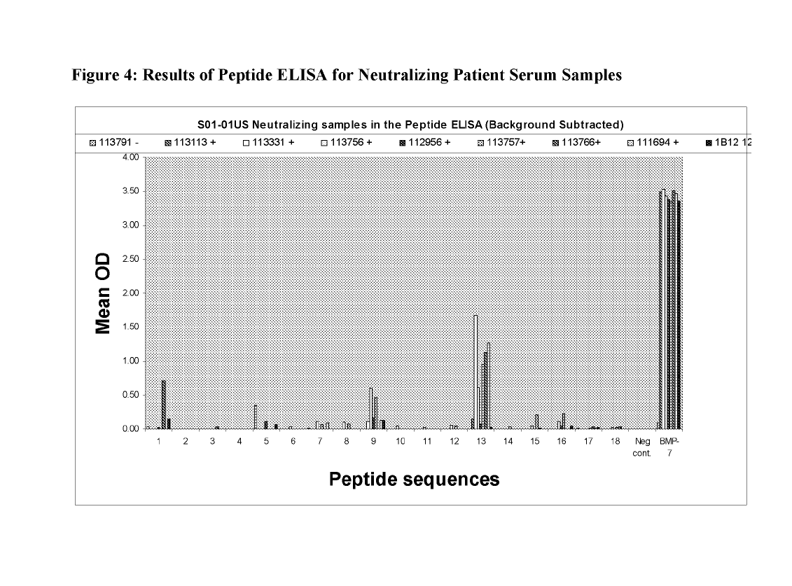

The results of binding of non-neutralizing anti-BMP-7 antibodies from patient serum to the 18 peptides is shown in FIG. 3 and the binding of neutralizing anti-BMP-7 antibodies from patient serum to the 18 peptides is shown in FIG. 4. As shown in FIG. 3, peptide 13 exhibited high binding affinity for the non-neutralizing anti-BMP-7 antibodies from the 3 positive patient serum samples (111694, 111945 and 111665) whereas negative patient serum (111266), as expected, showed no binding affinity. This indicates that peptide 13 contains a linear binding epitope for these non-neutralizing anti-BMP-7 antibodies.

As shown in FIG. 4, peptide 13 again exhibited a high binding affinity for neutralizing anti-BMP-7 antibodies from several of the positive patient samples, suggesting that peptide 13 contains a linear binding epitope for neutralizing anti-BMP-7 antibodies. Further, the data in FIG. 4 not only confirm binding of anti-BMP-7 antibodies to a linear epitope contained in peptide 13, but also suggest that neutralizing antibodies may have some binding affinity to a linear epitope contained in peptides 1 and 9 as well. Some binding was also observed for peptide 5.

### Example 2

### Engineering BMP-7 Proteins with Reduced Immunogenicity by Altering Epitopes

Peptide 9 has the amino acid sequence GYAAYYCEGECAFPL (SEQ ID NO:10), while Peptide 13 has the amino acid sequence VHFINPETVPKPACCA (SEQ ID NO:14). However, as shown in Example 1, epitopes lie in the regions of BMP-7 corresponding to peptide 9 and peptide 13. Accordingly, in order to reduce or eliminate the immunogenicity of these sequences, amino acid alterations are made in BMP-7 at residues corresponding to residues in peptide 9 and peptide 13.

In order to determine the specific residues responsible for the immunogenicity of peptides 9 and 13, several peptide analogs are generated wherein two consecutive amino acids are modified each to an alanine residue. Enough peptides are generated such that all permutations of peptides 9 and 13 with two consecutive alanine residues are created. The peptide analogs are then assayed for their ability to bind to anti-BMP7 antibodies in comparison to the ability of peptides 9 and 13 to bind anti-BMP-7 antibodies. Peptide analogs with decreased binding to anti-BMP-7 antibodies are identified and it is determined that one or more of residues 1, 3, 5, 6, 8, 10, or 12 of peptide 9 (corresponding to residues 61, 63, 65, 66, 68, 70, and 72 of mature human BMP-7 (SEQ ID NO:1)) and/or one or more of residues 2, 3, 4, 5, 6, 7, 8, 9, 10, 12, or 15 of peptide 13 (corresponding to residues 92, 93, 94, 95, 96, 97, 98, 99, 100, 102, or 105 of mature human BMP-7 (SEQ ID NO:1)) is responsible for binding to the anti-BMP-7 antibody and thereby causes the immunogenicity of recombinant human BMP-7. Accordingly, those residues determined as causing the immunogenicity of BMP-7 are modified, e.g., by substitution, to create a BMP-7 variant with reduced immunogenicity.

Alterations according to the invention, such as amino acid substitutions taught herein, are introduced into the genetic sequence for BMP-7 according to standard recombinant genetic engineering techniques. The variant BMP-7 is then expressed in a prokaryotic or eukaryotic expression system according to standard protocols. The expressed variant BMP-7 is then purified according to standard protocols.

### Example 3

### BMP-7 Variants Induce Alkaline Phosphatase Activity

The ability of BMP-7 variants of the invention to induce alkaline phosphatase (ALP) activity in the rat osteosarcoma cell line ROS 17/2.8 is assayed. Variant BMP-7s of the invention are tested in a nine point dose response in triplicate with wild-type BMP-7 used as a positive control. In particular, ROS 17/2.8 cells are plated in 96-well tissue culture plates. BMP-7 variants are added to the cells in the following dosages: 6000, 2000, 666, 222, 74, 24, 8, 2, and 0.9 ng/ml and incubated for a period of 48 hours. Cells are subsequently lysed and potency of the growth factors to induce ALP activity is assessed based on the EC50 derived from non-linear regression of the mean optical density (OD) of the samples. All of the BMP-7 variants tested demonstrate robust alkaline phosphatase activity, indicating that the variants maintain their biological activity.

### Example 4

### BMP-7 Variants have Reduced or Eliminated Immunogenicity Compared to Wild-Type

BMP-7 variants according to the embodiments of the invention are tested in primates to determine their immunogenicity. Eukaryotically produced BMP-7 variants are tested with wild-type human BMP-7 (eukaryotically produced) being administered as a control. In a typical experiment, rhesus macaques are injected with 40 μg/kg of the protein sample subcutaneously once a day for four weeks. At regular intervals, serum is obtained from the animals and serum concentrations of antibodies against BMP-7 are measured by ELISA using human IL-7 coated 96 well plates. Typically, serial dilutions of each serum sample are added to each well in triplicate for two hours, washed with 0.05% Tween (Tween 20) in PBS and blocked with 1% BSA/1% goat serum in PBS. To each sample, a horseradish peroxidase-conjugated anti-macaque IgG is added (1:60,000 in sample buffer), incubated at 37° C. for 2 hours, and the plates is washed 8 times with 0.05% Tween in PBS. Samples are then assayed using the colorimetric substrate solution OPD (o-phenylenediamine dihydrochloride) by measuring the OD at 490 nm, subtracting the background reading at 650 nm.

It is found that eukaryotically produced wild-type human BMP-7 gives rise to high anti-BMP-7 antibody titers. In contrast, the antibody titers of eukaryotically produced variant human BMP-7 give rise to significantly lower titers of anti-BMP-7 antibodies.

### Example 6

### Variant BMP-7 is Effective at Inducing Bone and Cartilage Growth in Low Concentrations in Human Patients

Two human patients each require treatment to effect posterolateral fusion in the lumbar spine. In one patient, 1.5 mg of variant BMP-7 in a matrix of bovine bone collagen and carboxymethylcellulose sodium (similar to OP-1® Putty, Stryker Biotech, Hopkinton, Mass.) is surgically implanted on each side of the spine at the site requiring fusion. The matrix is reconstituted with a sterile saline (0.9%) solution prior to implantation. In the other patient, 3.5 mg of wild-type BMP-7 in a matrix of bovine bone collagen and carboxymethylcellulose sodium (similar to OP-1® Putty, Stryker Biotech, Hopkinton, Mass.) is surgically implanted on each side of the spine at the site requiring fusion.

After a first period of several months, each patient's spine is viewed radiographically, for example, by X-ray to determine presence of bone growth at the fusion. In the patient receiving variant BMP-7, bone growth is detected at the fusion site. However, fusion is not complete. In the patient receiving wild-type BMP-7, the same level of bone growth is detected as in the patient receiving variant BMP-7. Again, fusion of the vertebrae is not complete.

After a second period of several months, equal to the first period of several months, each patient's spine is again viewed radiographically, for example, by X-ray. In each patient, fusion of the vertebrae at the site of implantation is complete.

Accordingly, variant BMP-7 may be administered in lower concentrations than the corresponding wild-type BMPs while still promoting the same rate of bone growth. This may be attributed to the lessened immune response to BMP-7 variants, thereby allowing lower concentrations of variant BMP-7 to be administered as compared to wild-type BMP-7 to achieve the same level of bone growth as no BMP-7 is lost to immune system response.

In another example, two human patients each require treatment to effect posterolateral fusion in the lumbar spine. In one patient, 3.5 mg of variant BMP-7 in a matrix of bovine bone collagen and carboxymethylcellulose sodium (similar to OP-1® Putty, Stryker Biotech, Hopkinton, Mass.) is surgically implanted on each side of the spine at the site requiring fusion. The matrix is reconstituted with a sterile saline (0.9%) solution prior to implantation. In the other patient, 3.5 mg of wild-type BMP-7 in a matrix of bovine bone collagen and carboxymethylcellulose sodium (similar to OP-1® Putty, Stryker Biotech, Hopkinton, Mass.) is surgically implanted on each side of the spine at the site requiring fusion.

After a first period of several months, each patient's spine is viewed radiographically, for example, by X-ray to determine presence of bone growth at the fusion. In the patient receiving variant BMP-7, bone growth is detected at the fusion site and the fusion of the vertebrae is complete. In contrast, in the patient receiving wild-type BMP-7, bone growth is detected at the site of implantation. However, fusion of the vertebrae is not complete.

After a second period of several months, equal to the first period of several months, the patient receiving wild-type BMP-7's spine is again viewed radiographically, for example, by X-ray. Fusion of the vertebrae at the site of implantation is complete.

Accordingly, variant BMP-7s may be administered in the same concentrations as the corresponding wild-type BMPs to achieve an accelerated rate of bone growth. This may be attributed to the lessened immune response mounted against BMP-7 variants, thereby permitting more of the variant BMP-7 than wild-type to facilitate bone growth.

1. A BMP-7 variant comprising at least 90% sequence identity with mature human BMP-7 (SEQ ID NO:1), wherein the BMP-7 variant comprises substitutions at one or more of the following positions corresponding to mature human BMP-7: G61, A63, Y65, Y66, E68, E70, A72, H92, F93, I94, N95, P96, E97, T98, V99, P100, P102 or A105.

2. The BMP-7 variant of claim 1, wherein the substitutions are one or more of the following: G61E, A63E/Q/H, Y65F/N, Y66E, E68D/K/H, E70G/D, A72S/F/P, H92N, F93N/L/S, 194M/K/S/V, N95V/F, P96S/N, E97S/T/D/K, T98K/I/S/Y/H, V991, P100G, P102A, or A105V.

3. The BMP-7 variant of claim 1, wherein the variant demonstrates BMP-7 activity.

4. The BMP-7 variant of claim 1, wherein the variant comprises at least 95% sequence identity with mature human BMP-7.

5. A nucleic acid encoding the BMP-7 variant of claim 1.

6. A recombinant expression vector comprising the nucleic acid of claim 1.

7. A cell comprising the expression vector of claim 6.

8. The cell of claim 7, wherein the cell is prokaryotic.

9. The cell of claim 7, wherein the cell is eukaryotic.

10. A composition comprising the BMP-7 variant of claim 1 and a pharmaceutical carrier.

11. A method of treating a skeletal disorder in a patient comprising administering to the patient a therapeutically effective amount of the BMP-7 variant of claim 1.

12. A method of reducing the immunogenicity of a human BMP-7 protein comprising the steps of:
identifying an immunogenic epitope in the amino acid sequence of human BMP-7; and
modifying the epitope by engineering one or more substitutions in the amino acid sequence of BMP-7, wherein the one or more substitutions occurs at any one or more of positions G61, A63, Y65, Y66, E68, E70, A72, H92, F93, I94, N95, P96, E97, T98, V99, P100, P102 or A105 corresponding to mature human BMP-7 to create a modified amino acid sequence.

13. The method of claim 12, wherein the one or more substitutions is any one or more of:
G61E, A63E/Q/H, Y65F/N, Y66E, E68D/K/H, E70G/D, A72S/F/P, H92N, F93N/L/S, 194M/K/S/V, N95V/F, P96S/N, E97S/T/D/K, T98K/I/S/Y/H, V991, P100G, P102A, or A105V.

14. The method of claim 12, further comprising the steps of expressing a protein encoded by the modified amino acid sequence in a suitable expression system and purifying the protein.

15. The method of claim 12, wherein expressing the protein occurs in a eukaryotic cell.

16. The method of claim 12, wherein expressing the protein occurs in a prokaryotic cell.


SEQUENCE LISTING (29 sequences)

SEQ ID NO: 1 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 15 aa, Homo sapiens
STGSKQRSQNRSKTP

SEQ ID NO: 3 - protein, 15 aa, Homo sapiens
QRSQNRSKTPKNQEA

SEQ ID NO: 4 - protein, 15 aa, Homo sapiens
KNQEALRMANVAENS

SEQ ID NO: 5 - protein, 15 aa, Homo sapiens
LRMANVAENSSSDQR

SEQ ID NO: 6 - protein, 15 aa, Homo sapiens
SSDQRQACKKHELYV

SEQ ID NO: 7 - protein, 15 aa, Homo sapiens
QACKKHELYVSFRDL

SEQ ID NO: 8 - protein, 15 aa, Homo sapiens
SFRDLGWQDWIIAPE

SEQ ID NO: 9 - protein, 15 aa, Homo sapiens
GWQDWIIAPEGYAAY

SEQ ID NO: 10 - protein, 15 aa, Homo sapiens
GYAAYYCEGECAFPL

SEQ ID NO: 11 - protein, 15 aa, Homo sapiens
YCEGECAFPLNSYMN

SEQ ID NO: 12 - protein, 15 aa, Homo sapiens
NSYMNATNHAIVQTL

SEQ ID NO: 13 - protein, 15 aa, Homo sapiens
ATNHAIVQTLVHFIN

SEQ ID NO: 14 - protein, 15 aa, Homo sapiens
VHFINPETVPKPCCA

SEQ ID NO: 15 - protein, 15 aa, Homo sapiens
PETVPKPCCAPTQLN

SEQ ID NO: 16 - protein, 15 aa, Homo sapiens
PTQLNAISVLYFDDS

SEQ ID NO: 17 - protein, 15 aa, Homo sapiens
AISVLYFDDSSNVIL

SEQ ID NO: 18 - protein, 15 aa, Homo sapiens
SNVILKKYRNMVVRA

SEQ ID NO: 19 - protein, 14 aa, Homo sapiens
KKYRNMVVRACGCH

SEQ ID NO: 20 - protein, 15 aa, Homo sapiens
GYHAFYCHGECPFPL

SEQ ID NO: 21 - protein, 15 aa, Homo sapiens
GYQAFYCHGDCPFPL

SEQ ID NO: 22 - protein, 15 aa, Homo sapiens
GYAAFYCDGECSFPL

SEQ ID NO: 23 - protein, 15 aa, Homo sapiens
GYAANYCDGECSFPL

SEQ ID NO: 24 - protein, 15 aa, Homo sapiens
EYEAYECKGGCFFPL

SEQ ID NO: 25 - protein, 14 aa, Homo sapiens
VNSVNSKIPKACCV

SEQ ID NO: 26 - protein, 14 aa, Homo sapiens
VNSVNSSIPKACCV

SEQ ID NO: 27 - protein, 15 aa, Homo sapiens
VHLMFPDHVPKPCCA

SEQ ID NO: 28 - protein, 15 aa, Homo sapiens
VHLMNPEYVPKPCCA

SEQ ID NO: 29 - protein, 15 aa, Homo sapiens
VHLKFPTKVGKACCV